(19) United States
(12) Patent Application Publication
(10) Pub. No.: US 2025/0299468 A1
(43) Pub. Date: Sep. 25, 2025

(54) SYSTEM AND METHODS FOR COMBINED REAL-TIME AND NON-REAL-TIME DATA PROCESSING

(71) Applicant: PULSEMEDICA CORP., Edmonton (CA)

(72) Inventors: Nir Katchinskiy, Edmonton (CA); Christopher Ceroici, Edmonton (CA); Geoffroy Rivet-Sabourin, Edmonton (CA)

(21) Appl. No.: 18/863,289

(22) PCT Filed: May 5, 2023
(86) PCT No.: PCT/CA2023/050619

§ 371 Date: Nov. 5, 2024

(30) Foreign Application Priority Data
May 6, 2022 (CA) 3157811

(51) Int. Cl. G06V 10/62; G06V 10/77; G06V 10/94; G06V 40/18; G16H 20/40; G16H 30/40

(52) U.S. Cl. CPC G06V 10/62; G06V 10/77; G06V 10/94; G06V 40/193; G16H 20/40; G16H 30/40; G06V 2201/03

(57) ABSTRACT

Features can be tracked across frames. The features are first identified in an initial frame using an image processing technique which may take a relatively long time to complete, such as the length of several frames. When the features are being identified within the initial frame, subsequent frames are stored in a fast track buffer and once the features are identified in the initial image, the features can be tracked across the frames in the fast track buffer using a relatively fast process, such a one that is able to process the buffer at a higher frame rate than the frames are received at.

### RELATED APPLICATIONS

The current application claims priority to Canadian application 3,157,811 filed May 6, 2022 entitled “SYSTEM AND METHODS FOR COMBINED REAL-TIME AND NON-REAL-TIME DATA PROCESSING,” The entire contents of which are incorporated herein by reference in their entirety.

### TECHNICAL FIELD

The current disclosure relates to data processing and in particular to data processing using combined real-time and non-real-time processing.

### BACKGROUND

Images can be processed to identify features within the image. Identifying features within an image can take a length of time that make real-time identification of feature difficult without having access to large computational resources.

Image processing techniques can be used in various treatment processes. For example, a patient's eye may be imaged and locations requiring treatment, such as by a treatment laser can be identified. The identified treatment location may then be treated by a therapeutic laser or using robotic surgery techniques.

Additional, alternative and/or improved techniques for processing data, such as image data, are desirable.

### SUMMARY

In accordance with the present disclosure there is provided an image processing method comprising: receiving a first image of an image stream having a stream frame rate; passing the received first image to image processing functionality; receiving a plurality of subsequent images of the image stream; storing the received plurality of subsequent images in a fast-track image buffer; subsequent to receiving at least one subsequent image, receiving from the image processing functionality an indication of one or more features within the first image; and tracking the one or more features across the plurality of subsequent images stored within the fast-tracking image buffer using a tracking process configured to process each of the subsequent images at a processing frame rate higher than the stream frame rate.

In a further embodiment of the method, the image processing functionality identifies the one or more features in the received first image.

In a further embodiment of the method, the image processing functionality identifies the one or more features in the received first image using a machine learning process.

In a further embodiment of the method, passing the received first image to the image processing functionality comprises: passing the first image to the processing functionality implemented at a remote computing device over a communication interface.

In a further embodiment of the method, the method further comprises: after tracking the one or more features across all of the subsequent images stored in the fast-tracking buffer, tracking the one or more features across newly received images of the image stream.

In a further embodiment of the method, tracking the one or more features across the newly received images of the image stream uses the tracking process used for tracking the one or more features across the subsequent images stored within the fast-tracking image buffer.

In a further embodiment of the method, tracking the one or more features across the newly received images of the image stream uses a different tracking process than used for tracking the one or more features across the subsequent images stored within the fast-tracking image buffer, wherein the tracking process for tracking the one or more features across the newly received images of the image stream has a slower processing frame rate than the processing frame rate of the tracking process used for tracking the one or more features across the subsequent images stored within the fast-tracking image buffer.

In a further embodiment of the method, the method further comprises: receiving new images of the image stream while tracking the one or more features across the subsequent images stored within the fast-tracking image buffer; and storing the new images in the fast-tracking image buffer.

In a further embodiment of the method, the method further comprises one or more of: removing subsequent images stored in the fast-tracking image buffer once processed by the tracking process; and marking subsequent images stored in the fast-tracking image buffer as safe for removal once processed by the tracking process.

In a further embodiment of the method, image stream is of a patient's eye, the method further comprising: using the one or more identified features tracked across images of the image stream in treatment of an eye condition.

In accordance with the present disclosure there is provided an image processing device comprising: a processor capable of executing instructions; and a memory storing instructions which when executed by the processor configure the image processing device to provide a method comprising: receiving a first image of an image stream having a stream frame rate; passing the received first image to image processing functionality; receiving at least one subsequent image of the image stream; storing the received at least one subsequent images in a fast-tracking image buffer; subsequent to receiving at least one subsequent image, receiving from the image processing functionality an indication of one or more features within the first image; and tracking the one or more features across the subsequent images stored within the fast-tracking image buffer using a tracking process capable of processing the subsequent images at a processing frame rate higher than the stream frame rate.

In a further embodiment of the device, the image processing functionality identifies the one or more features in the received first image.

In a further embodiment of the device, the image processing functionality identifies the one or more features in the received first image using a machine learning process.

In a further embodiment of the device, the device further comprises a communication interface for communicating with a remote computing device, the method provided by execution of the instructions comprises: passing the first image to the processing functionality implemented by the remote computing device over the communication interface.

In a further embodiment of the device, the communication interface is at least one of: a PCIe communication interface; a USB interface; a Bluetooth interface; a wired network interface; and a wireless network interface.

In a further embodiment of the device, the method provided by executing the instructions further comprises: after tracking the one or more features across all of the subsequent images stored in the fast-tracking buffer, tracking the one or more features across newly received images of the image stream.

In a further embodiment of the device, tracking the one or more features across the newly received images of the image stream uses a same tracking process as used for tracking the one or more features across the subsequent images stored within the fast-tracking image buffer.

In a further embodiment of the device, tracking the one or more features across the newly received images of the image stream uses a different tracking process than used for tracking the one or more features across the subsequent images stored within the fast-tracking image buff, wherein the tracking process for tracking the one or more features across the newly received images of the image stream has a slower processing frame rate than the processing frame rate of the tracking process used for tracking the one or more features across the subsequent images stored within the fast-tracking image buffer.

In a further embodiment of the device, the method provided by executing the instructions further comprises: receiving new images of the image stream while tracking the one or more features across the subsequent images stored within the fast-tracking image buffer; and storing the new images in the fast-tracking image buffer.

In a further embodiment of the device, the method provided by executing the instructions further comprises one or more of: removing subsequent images stored in the fast-tracking image buffer once processed by the tracking process; and marking subsequent images stored in the fast-tracking image buffer as safe for removal once processed by the tracking process.

In a further embodiment of the device, image stream is of a patient's eye, the method further comprising: using the one or more identified features tracked across images of the image stream in treatment of an eye condition.

### BRIEF DESCRIPTION OF THE DRAWINGS

Further features and advantages of the present disclosure will become apparent from the following detailed description, taken in combination with the appended drawings, in which:

FIG. 1 depicts a hardware controller configured for tracking image features across frames;

FIG. 2 depicts a process for tracking image features across frames;

FIG. 3 depict a method for tracking image features across frames;

FIG. 4 depicts a system for distributed tracking of image features across frames;

FIG. 5 depicts a further process for tracking image features across frames;

FIG. 6 depicts illustrative graphic user interfaces;

FIG. 7 depicts an ophthalmological laser imaging and treatment system;

FIG. 8 depicts a method of hybrid data processing; and

FIG. 9 depicts a further method of hybrid data processing.

### DETAILED DESCRIPTION

A hybrid data processing approach is described further herein that uses both real-time and non-real-time processing techniques. During the non-real-time processing, the real-time data may be captured and stored in a buffer and the results of the non-real-time processing may be applied to the buffered data in a manner that allows the non-real-time processing results to catch up with the real-time processing. The non-real-time processing may allow more complex processing including, for example, processing using machine learning (ML) techniques. ML processing techniques maybe more computationally expensive and so require longer to process data. The hybrid data processing is described further below with particular reference to image processing, however, similar techniques may be applied to other types of data.

The hybrid processing technique may allow a real-time processing technique to be used for example to track eye movement and ensure a treatment process is done accurately. While the real-time processing can ensure treatment is performed accurately, a non-real-time process can be performed in parallel to perform other tasks, such as identifying additional, or next treatment targets, evaluate the performance of the treatments, etc. Although, the non-real-time process may be relatively slow, it may still be performed within a length of time during which the treatment intervention is being performed so that the processing results may be available before the intervention is completed.

Image processing tasks can be performed on images. The images may be frames/pixels/lines/subframes of an image stream or video that are captured at a particular processing rate. If the image processing, such as feature or object detection, occurs at a processing rate that is lower than the image stream or video frame rate, the image stream or video cannot be processed in real-time. Image processing methods and systems described in further detail below may process an initial image frame, or parts of the image frame such as a subframe, line, or group of pixels using an image processing technique that has a lower processing rate than the image stream or video frame rate. As the initial image is being processed, the system stores subsequent images in a buffer. Once the image processing is completed on the initial image, for example to identify particular features, and/or objects within the image, the image processing results, for example the identified features and/or objects, can be quickly tracked across the images in the buffer. The image processing results/features can be tracked using a process that has a processing rate higher than the frame rate allowing the buffer to be emptied. Once the features have been tracked across all of the images in the buffer, the features can continue to be tracked across images as they are received.

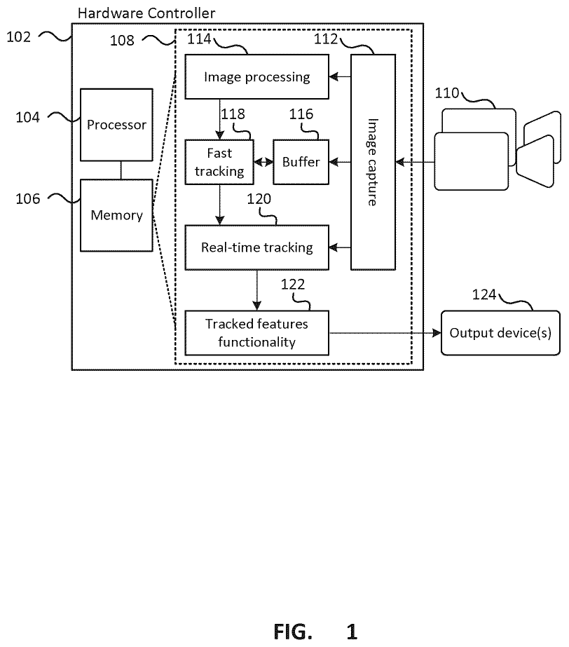

FIG. 1 depicts a hardware controller configured for tracking image features across frames. The hardware controller 102 includes a processor 104 and memory 106. The memory 106 may store instructions which when executed by the processor 104 configure the hardware controller to provide various functionality 108. Additionally or alternatively, the functionality 108 may be provided, at least in part, by a field programmable gate array (FPGA) or an application specific integrated circuit (ASIC) of a digital signal processor (DSP) or a microcontroller (MCU) or a processor (CPU). The hardware controller 102 may be connected to one or more image capture devices 110. For example, the hardware controller may be used as part of an ophthalmological device and the image capture devices may comprise a Scanning Laser Ophthalmoscopy (SLO) device and/or an Optical Coherence Tomography (OCT) device. When multiple imaging devices are present, the imaging devices may be registered with each other so that they each capture a common portion or location of the imaging subject.

The functionality 108 of the hardware controller 102 comprises image capture functionality that receives, or retrieves, images from the image capture device(s) 110. The Image capture functionality 108 captures images and provides them, or makes them available to other functionality. The images may be captured at a particular frame rate to provide an image stream, subframe stream, line steam, pixel stream or video. The image capture functionality 112 may also reformat and/or re-encode, the captured images to a different format used by other components of the hardware controller 102.

The hardware controller 102 may include image processing functionality 114 that processes a captured image. The image processing functionality 114 may for example identify particular features within the image, possibly using artificial intelligence techniques and/or machine learning (ML) techniques. The image processing functionality 114 takes a finite amount of time to complete and when the processing time, or processing rate, is longer than the time between captured images, or the frame rate, the image processing cannot identify features in images in real time. When the processing time, or processing rate, is equal to or shorter than the time between captured images, or the frame rate, the processing can occur in real-time, or faster than real time. Depending upon the computational resources available at the hardware controller, a number of images may be received while the initial frame is being processed. These additional frames may be stored in a buffer 116 on the hardware controller 102 as they are received or captured. Once the image processing is completed, for example by identifying features within the initial image, fast tracking functionality 118 tracks the processing results, or identified features, across the frames in the buffer 116. Once the features are identified by the image processing functionality 114 the fast tracking functionality 118 may track the features using various tracking techniques including for example, the use of sum of squared differences (SSD), Kalman filters, optical flow, deep learning method, etc. Once the fast tracking functionality 118 has tracked the features across all of the images stored in the buffer 116, the features can be tracked across newly received/captured images using real-time tracking functionality 120. Although depicted separately in FIG. 1 the fast-tracking functionality 118 and the real-time tracking functionality 120 may use the same or different tracking techniques. Both the fast-tracking functionality 118 and the real-time tracking functionality may be provided by the same functionality. Further, it is possible that the real-time tracking functionality may retrieve or access the images from the buffer as they are captured and stored instead of being retrieved from the image capture functionality 112.

The real-time tracking functionality 120 provides feature locations within images as they are captured. The feature locations can be provided to some functionality 122 that makes use of the tracked features. As an example, the tracked features functionality 122 may output the features on output device(s) 124. The tracked features functionality 122 may simply display the features on the captured images on a display or monitor, or may comprise more complex functionality such as generating a treatment plan for treating one or more of the tracked features. Additionally, the tracked features may allow for the real-time tracking of eye movement as well as compensating other data, such as a treatment plan, for the eye movement. The features may provide a reference, or system of coordinates, to account for eye movement. The real-time tracking for eye movement may be done using various techniques, including for example the fast retina tracking technique described in Canadian patent application 3,135,405 filed Oct. 22, 2021 entitled “FAST RETINA TRACKING” the entire contents of which are incorporate herein by reference in their entirety.

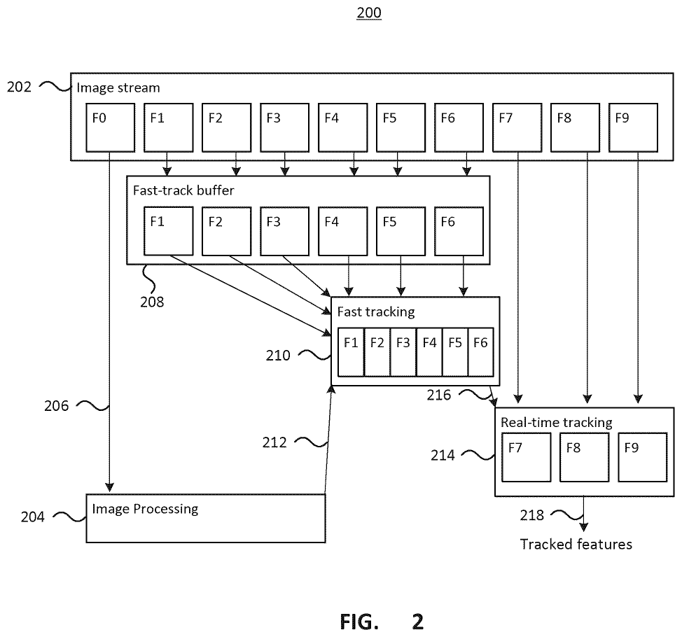

FIG. 2 depicts a process for tracking image features across frames. The process 200 may be performed by, for example, a hardware controller such as that depicted in FIG. 1. As depicted an image stream 202 may comprise a time-series of frames F0 . . . F9 captured at a particular frame rate. The initial frame, F0, is captured and passed to image processing functionality 204 as depicted by arrow 206. The image processing functionality 204 performs image processing on the frame F0, which takes some length of time. While the initial frame is being processed by the image processing functionality 204, additional image frames are captured and stored in fast-track buffer 208. For example, frames F1, F2 and F3 may be captured while the image processing is still being performed. Once the image processing on the initial frame F0 is complete, the results are passed to fast tracking functionality 210 as depicted by arrow 212. The fast tracking functionality 210 retrieves the frames from the buffer and tracks the features identified by the image processing functionality across the images in the buffer 208. As depicted, the fast tracking process 210 takes a finite amount of time to track the features across each image; however, the fast tracking functionality can track the features at a processing rate greater than the frame rate of the image stream. As the features are being tracked across the image frames stored in the buffer, additional frames, such as frames F4, F5, and F6, that are received may be stored in the buffer for further processing by the fast tracking functionality. Although depicted as a linear buffer, the buffer may be a circle buffer that can provide buffering for a certain length of time or number of frames. The circular buffer may be useful in scenarios where new images do not have any features and as such can be written over with further images. Additionally, the circular buffer may be useful in scenarios when additional objects or features do not need to be tracked across buffered frames.

Once the fast tracking functionality 210 has processed all of the images in the fast-track buffer, the features of the last image, depicted as image frame F6 in FIG. 2, may be passed to real-time tracking functionality 214 s depicted by arrow 214. The real-time tracking functionality 214 uses the tracked features to continue the tracking of the features across frames as they are received. As depicted, frames F7, F8 and F9 can be provided to the real time tracking functionality as they are received. As the feature tracking is completed on each frame, the tracked features can be output from the real-time tracking functionality 214 as depicted by arrow 218. The tracked features provided from the real-time tracking functionality 214 may be used for various applications, including for example, in tracking treatment locations within a patient's eye for treatment with a therapeutic laser.

Although the above describes processing the first frame, F0, of the image stream by the image processing functionality 204, it is possible to carry out the image processing, and subsequent fast feature tracking by the fast tracking functionality, on other frames. For example, rather than processing a single frame at a time, a group of frames may be provided. Additionally, the feature tracking may be performed periodically in order to update the features being tracked. Additionally or alternatively, the image processing may be performed if/when the feature tracking fails, which may occur if there is too much movement between frames, or when an action is occurs or is completed.

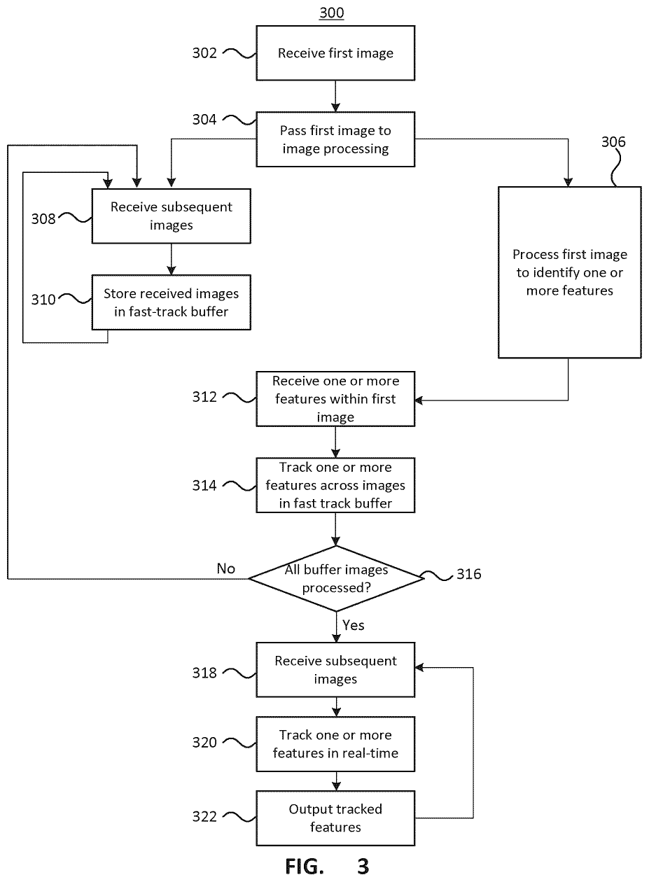

FIG. 3 depicts a method for tracking image features across frames. The method 300 receives a first image (302). Although depicted as a first image, it could be a set of images, one or more subframes or pixels, etc. The first image may be an image frame within an image stream captured from an image capture device at a particular frame rate, for example at 10 frames per second (fps), 20 fps, 30 fps, or other frame rates. The first frame is passed to image processing functionality (304) and is processed to identify one or more features/objects within the image (306). The image processing functionality processes the first image at a processing rate that is less than the frame rate of the image stream. While the image processing is being performed on the first image, a subsequent image of the image stream is received (308) and stored in a fast track buffer (310). While the first image is still being processed by the image processing functionality, the subsequent images that are received continue to be stored in the fast-track buffer (310). Once the first image is processed, one or more features are received, retrieved or otherwise provided (312) and the at least one or more features tracked across the images stored in the fast track buffer (314). The feature tracking across the images stored in the fast track buffer is done at a processing rate that is greater than the frame rate of the image stream. Although the one or more features are described as being provided once the processing of the first image is complete, it is possible that the one or more images are provided as they are identified within the image and the image processing may continue. While the one or more features are tracked across the images in the buffer, any images of the image stream that are received are again stored in the fast-track buffer (310), that is while there are still images in the fast track buffer that have not been processed (No at 316) subsequently received images are added to the buffer. When there are no more images in the fast track buffer (Yes at 316) subsequent images of the image stream are received (318) and the one or more features tracked across the newly received feature in real-time (320), or at a processing rate that is greater than the frame rate of the image stream. As the features are tracked across newly received images the tracked features and their locations may be output for use by additional functionality (322).

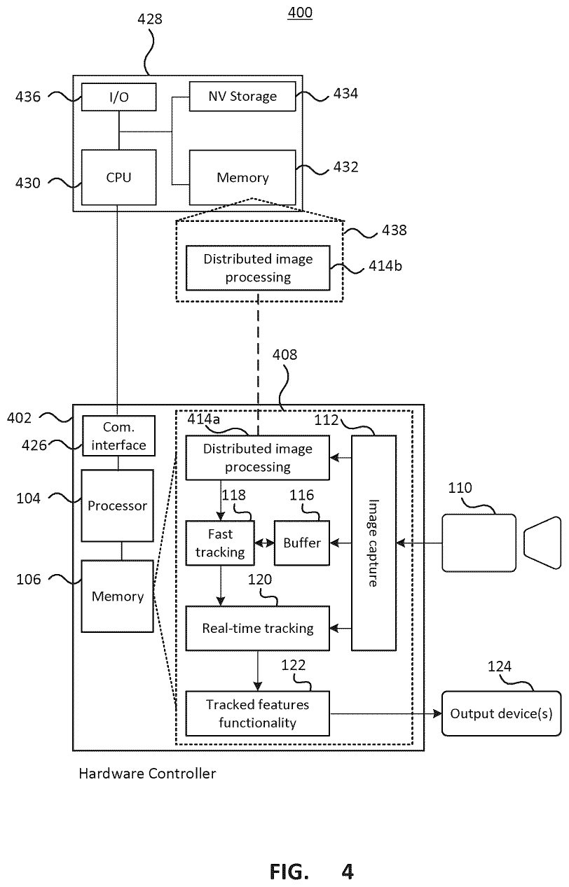

FIG. 4 depicts a system for distributed tracking of image features across frames. The system 400 is similar to the hardware controller described above, however the image processing is done in a distributed manner. Similar components to those in FIG. 1 have the same reference numbers and are not described in further detail. The system 400 comprises a hardware controller 402, which is similar to the hardware controller 102 described above with reference to FIG. 1. The hardware controller 402 may comprise a processor 104 and a memory 106 that provide various functionality 408. The hardware controller 402 may be connected to one or more image capture device 110 which provide image data to image capture functionality 112. The image may be passed to distributed image processing functionality 414a on the hardware controller 402. The distributed image processing functionality 414a operates in cooperation with distributed image processing functionality 414b provided on a separate device from the hardware controller. The distributed image processing functionality 414a, 414b may provide similar functionality as the image processing functionality described above with reference to FIG. 1. The distributed image processing functionality 414a on the device may perform some of the image processing or may simply pass the images to the distributed image processing functionality 414b on the separate processing device 428. The hardware controller 402 may include a communication interface 426 for communicating with a separate processing device 428 that provides the distributed image processing functionality 414b.

The separate processing device 428 may include a processing unit 430, and memory 432. The device 428 may further include non-volatile storage 434 and one or more input/output (V/O) interfaces for connecting the separate computing device to other device, including for example, the communication interface of the hardware controller. The communication between the hardware controller and the separate processing device may be provided using various different technologies including for example network communications such as TCP/IP, serial communication including for example USB, SCSI communication, PCIe communication, etc. The memory 432 may store instructions which when executed by the processor configure the separate computing device to provide functionality 438 including the distributed image processing functionality 414b.

Providing the image processing functionality on a separate device may provide greater computational resources for processing the image/images faster compared to the image processing of the hardware controller described above. The distributed processing of the hybrid approach described herein may provide additional computational resources that are well suited for parallel processing tasks, or performing multiple different processing tasks. While the image processing may be performed faster on the separate processing device 428, the overall processing time, from when the image is received by the distributed image processing functionality on the hardware controller 402 to when the processing results are received back at the distributed image processing functionality 414a on the hardware controller, may still be relatively long, compared to the frame rate of the image stream and as such a number of image frames may be received while the image is being processed. The received images are stored in a buffer 116, and fast tracking functionality can be applied to the stored images once the features are received from the distributed image processing functionality. Similar to the process described above, once the features have been tracked across all of the images stored in the buffer 116, subsequently received images can be processed by the real-time tracking functionality 120 and the results provided to some functionality 122 that makes use of the tracked features, which may be provided to one or more output device 124.

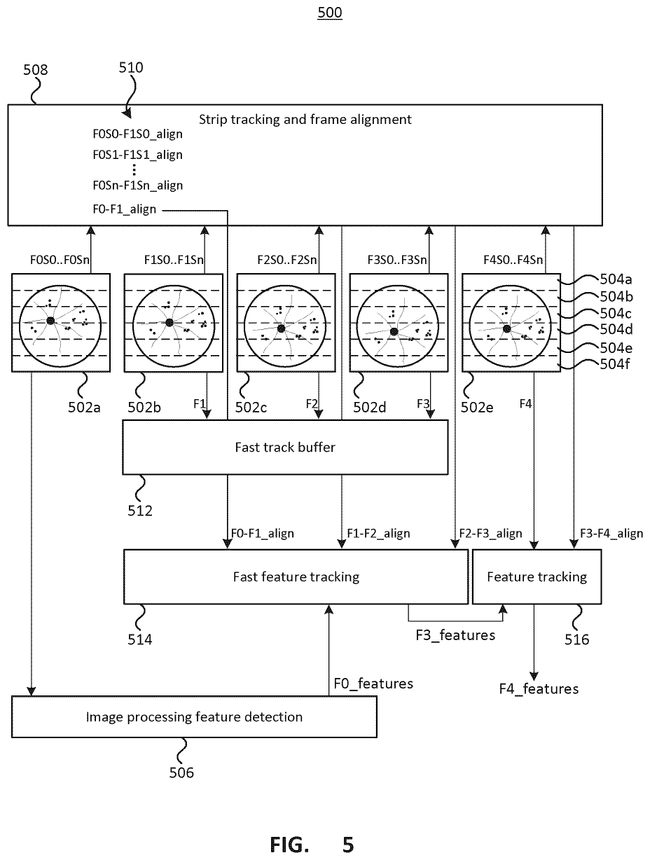

FIG. 5 depicts a further process for tracking image features across frames. The process depicted in FIG. 5 uses the feature tracking described above to track features within images of a patient's eye that can be targeted for treatment for example by a therapeutic laser. As described further below, the feature tracking may work in conjunction with retina tracking that adjusts frame alignment or registration to adjust for eye movement. Although not depicted in FIG. 5, the captured images, or a representation of the captured images, may be stored for further processing or review.

As depicted, a number of frames 502a . . . 502e capture images of a patient's eye. Although eye movement is restricted during treatment, there may still be some eye movement which should be accounted for to ensure laser treatments are targeted at the desired locations. In addition to ensure a possible laser treatment targets the desired locations, the tracking of eye movement may be useful or necessary in order to be able to track objects or features within the eye. The images may be captured as a single frame or may be captured as a plurality of rows that are combined together. As depicted, each of the frames may comprise a number of strips 504a . . . 504f, that are combined together into the individual frame. As each strip of each frame is captured, it can be provided to strip tracking and frame alignment functionality. The strip tracking and frame alignment functionality may perform retina tracking not only between complete frames, but also on each strip, which allows for fast retina tracking to ensure any targeted locations can be correctly targeted.

When an initial frame 502a is received, it can be provided to image processing functionality 506 that processes the frame to identify certain features within the image. As an example, the feature detection may identify features associated with a medical condition such as floaters, drusen associated with age-related macular degeneration (AMD), cataracts, microaneurysms associated with diabetic retinopathy, glaucoma, etc. The feature detection takes some length of time to perform, during which additional frames of the patient's eye are captured.

As strips of frames are captured, they can be provided to strip tracking and alignment functionality 508. Each frame is identified in FIG. 5 as Fx, where x represents the frame number, and each strip is identified as Sy, where y represents the strip number. For example F1S3 identifies the 4th strip of the second frame, since the first frame and first strip is F0 and S0 respectively. The strip tracking and alignment functionality can determine an alignment 510 that includes strip alignments between strips depicted as F0S0 . . . n−F1S0 . . . n_align, which may provide an indication of for example a translation representing the eye movement, as well as a full frame alignment between the complete frames depicted as F0-F1_align, which may provide translations and rotations representing the patient's eye movement between full frames, or possibly between a current frame and a reference frame. During treatment, the sub-frame alignment provided by the strip tracking and the full frame alignment can be used to ensure target locations for laser treatment account for the patient's eye movement.

While the initial image frame 502a is being processed by the image processing functionality, additional frames are received and stored in a fast track buffer 512. The image processing feature detection identifies feature locations within the initial frame, depicted as F0_features, and the features are provided to fast feature tracking functionality 514 which tracks the identified features across the images stored in the fast track buffer 512. The fast track feature tracking may use the frame alignment information, depicted a F0-F1_align, F1-F2_align, and F2-F3_align, from the strip tracking and frame alignment functionality.

Once the fast feature tracking functionality has tracked the features across all of the features in the buffer, the feature locations can continue to be tracked across newly received features in real-time 516 and the features output, depicted as F4_features. The feature tracking 516 may also use the frame alignment information to account for eye movement while tracking features across the images. The feature locations within an image may be used, in conjunction with the sub-frame strip alignment information, for targeting a treatment laser.

The tracking described above can track a patient's eye movements within a single frame using the strip or sub-frame tracking. The tracking information may be used by other imaging processing functionality. For example, the image tracking may be used to provide image stabilization across multiple frames which may make tracking other features such as floaters that move easier.

The above has described applying the feature detection to an initial image. It is possible to apply the feature tracking to additional images periodically to possibly identify features not in the initial image as well as possibly correct and/or verify the feature locations being tracked. Further, the image processing may be performed if or when the feature tracking fails. Further still, the image processing may be performed when an action is performed or completed. For example, when treating an ocular condition, the image processing may be performed after an area is treated with a laser in order to evaluate the treatment.

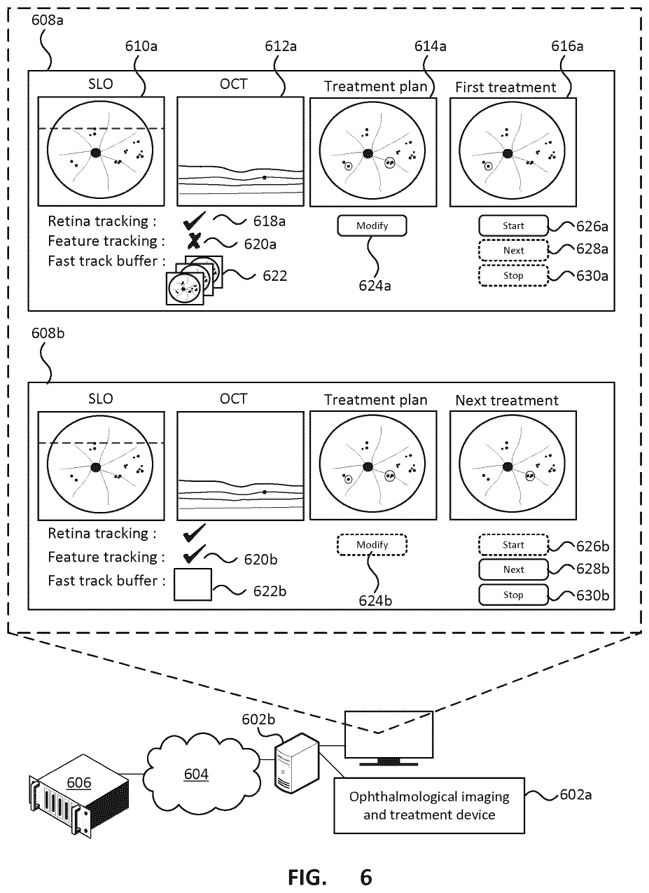

FIG. 6 depicts illustrative graphic user interfaces. An ophthalmological imaging and treatment device 602a, which may include a hardware controller including the feature detection and tracking functionality described above, may be coupled to computing device 602b and may provide a graphical user interface (GUI) for display to users. Illustrative screens 608a, 608b of the GUI are depicted, however these are intended only to provide examples of possible interfaces. In addition to providing the GUI, the imaging and treatment device 602a and/or the computing device 602b may be coupled to one or more networks 604 to communicate with remote computing devices 606, which may provide various functionality. For example, the remote computing device 606 may provide distributed image processing functionality, and/or additional services or functionality such as data storage, and/or remote user interfaces.

The screen 608a depicts an initial screen while features are being identified. The interface may provide one or more images from the imaging devices, depicted a SLO image 610a and a corresponding OCT image 612a taken along a location depicted by the broken line in the SLO image. Although depicted as SLO and OCT imaging devices, different imaging modalities may be provided. Additionally, the GUI 608a may present a treatment plan 614a that may have been previously developed specifying treatment locations depicted as circles in the treatment plan. The screen 608a may also provide an indication of the first treatment location 616a from the treatment plan that will be targeted once treatment begins. The screen 608a may also provide information about the status of processes. For example, it may provide an indication that the retina tracking is being performed successfully 618a. In screen 608a the feature tracking is depicted as not being performed 620a as the features are still being identified. The screen may also provide an indication of the buffer 622 used for the fast tracking of the features, once available. Additionally, the GUI may provide one or more components for interacting with the system, including for example, a button 624a for modifying the treatment plan, a button 626a for starting the treatment, a button for moving to the next treatment location 628a and a button for stopping or pausing the treatment 630a. Some buttons or components may be active or inactive depending on the operational state of the system. For example, the ‘next’ and ‘stop’ buttons may be inactivated since the treatment has not yet started.

The hybrid processing using real-time and non-real-time processing described above may use the real-time processing to, for example, track eye movement and update treatment locations based on the eye movement, while the non-real-time processing may provide various functionality including for example feature identification if the tracking process loses features as well as other functionality such as a ML process that can process the results of treatment and adjust the treatment including suggesting other treatments that could be performed at this time or possibly stopping the current treatment. If a new treatment option is provided,

The screen 608b is similar to the screen 608a; however, it is assumed that the treatment process has started. As depicted, the feature tracking is being performed successfully 620b and the buffer is empty 622b, so the feature tracking is being done in real time. Additionally, the buttons that are active or inactive may be adjusted. As depicted, the button for modifying a treatment plan 624b may be inactive while the treatment plan is being carried out. Similarly, once the treatment has started, the ‘start’ button may be inactive 626b, while the ‘next’ button 628b for moving to the next treatment location and the ‘stop’ button 630b for stopping or pausing the treatment may both be active.

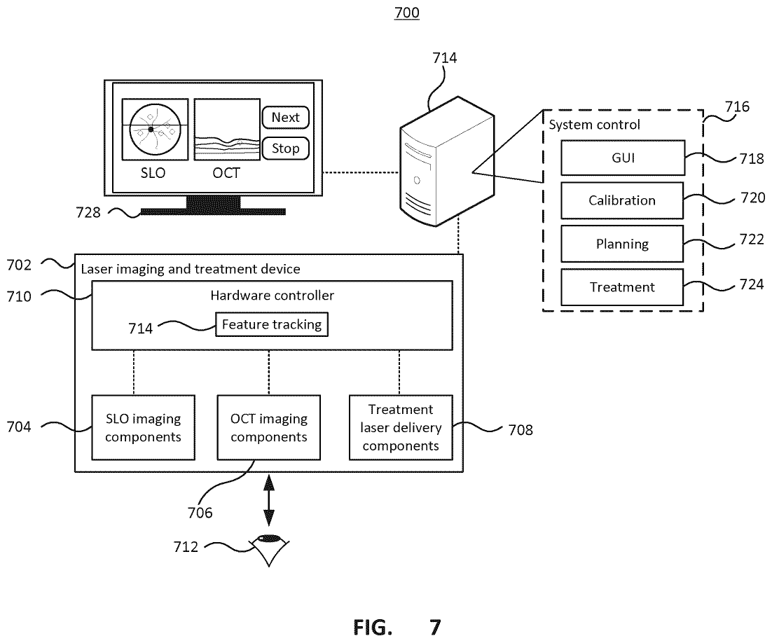

FIG. 7 depicts an ophthalmological laser imaging and treatment system. The system 700 comprises an imaging and laser delivery device 702. The device 702 comprises SLO imaging components 704, OCT imaging components 706 and treatment laser delivery components 708. The imaging and laser delivery components may be controlled by a hardware controller 710. The light for the SLO imaging, OCT imaging and treatment laser may be delivered to an eye 712, or possibly other target, being imaged and/or treated. The imaging light for SLO and OCT imaging is reflected back to the respective detectors.

The device controller 710 may provide an interface between the device 702 and a computing device 714. The computing device 714 provides various system control functionality 716 for operating the imaging and laser delivery device 702. While the computing device 714 is depicted as a separate computing device 714, it is possible to incorporate the computing device 714, or possibly one or more of the components provided by the computing device, into the imaging and laser delivery device 702. The hardware controller 710 may capture signals from respective detectors/camera of the SLO, and OCT imaging components 704, 706 as well as controlling other components, such as the sources of the imaging components, 704, 706, and treatment laser delivery components 708, focusing components, or other components. As depicted, the hardware controller 714 may include feature tracking functionality 714 described above. The feature tracking functionality may be performed completely by the hardware controller, or may be performed in a distributed manner in cooperation with the computing device 714, or other remote devices (not shown).

The computing device 714 may comprise one or more processing units (not depicted) for executing instructions, one or more memory units (not depicted) storing data and instructions, which when executed by the one or more processing units configure the computing device to provide the system control functionality 716. The system control functionality 716 may include graphical user interface (GUI) functionality 718 that provides a GUI for operating the imaging and laser delivery device. Calibration functionality 720 may be provided in order to calibrate the imaging and laser delivery device 702 and in particular to register and correlate the SLO imaging components 704, OCT imaging components 706 and the treatment laser delivery components 708 so that locations in the SLO images and OCT images can be precisely registered with each other and be accurately targeted by treatment laser. Image processing techniques may be used to co-register different components, or the components may be re-using other techniques such as co-registering different systems such as the treatment laser and OCT imaging components using various sensors and actuators to physically register the two components with each other. Planning functionality 722 may be provided that allows a treatment plan to be developed for treating a particular ocular condition. The planning functionality 722 may use the GUI functionality to allow a user to define the treatment plan. The planning functionality 722 may allow planning for various different conditions or different planning functionality may be provided for different conditions. Additionally or alternatively, the planning functionality may incorporate automated, or semi-automated, planning functionality that may identify treatment locations within the captured images. The planning functionality 722 may also continually process captured images and/or other data collected from sensors such as an Intraocular pressure (IOP) sensor, and may adjust the treatment plan based on the processing. The treatment planning may be performed in a non-real-time manner and then fast tracked to the current time using the buffered data as described above. Treatment functionality 724 may control the components of the device 702, including the treatment laser delivery components 708, in order to carry out the treatment plan in order to treat, or at least partially treat, an ocular condition.

The GUI functionality 718 may present the generated GUI on a display device 728. Although depicted as a separate display, the display could be incorporated into the imaging and laser delivery device 702. Although the GUI presented may vary depending upon what information needs to be, or may be desirable to be, displayed to the user. FIG. 7 depicts a GUI that could be displayed during treatment. For example, the GUI may display a SLO image, and an OCT image. The SLO image may include an indication of the location of the cross section of the OCT image. The SLO image, and the OCT image may include indications of treatment locations that have not yet been treated as well as treatment locations that have been treated. The GUI may include other details of the treatment plan that may be relevant to the user as well as graphical elements for starting/stopping the treatment or proceeding with the treatment such as proceeding to the next treatment location.

The system 700 may be used for imaging eyes to identify areas for treatment and carrying out the treatment. The treatment may be for a wide range of different ocular conditions including, for example, floaters, age-related macular degeneration (AMD), vitreomacular traction syndrome (VMT), diabetic retinopathy, cataracts, choroidal neovascularization, microaneurysm, glaucoma, epiretinal membrane (ERM), retinal tears and detachment, central or branch vein occlusion.

The systems and methods described above have described identifying features in an image and then tracking the features across buffered frames. It is possible that the feature identification and tracking across buffered images may be done in a plurality of different imaging modalities. For example, the feature identification and tracking may be applied to both SLO images and OCT images using respective feature identification algorithms and image buffers.

The above has described a hybrid approach to image processing that allows a relatively slow image processing technique to be used along with real-time image processing. For example, the initial feature detection process may not be performed in real-time however once the features are identified, they may be tracked across frame faster than real-time allowing the feature tracking to be applied to buffered frames in order to “fast-forward” the image tracking. While the hybrid processing has been described above with particular reference to image processing and feature detection and tracking, a similar approach may be used to allow relatively slow processing techniques to be used along with relatively fast processing techniques.

For example, the slow processing technique may use machine learning or artificial intelligence based processing techniques. The processing may use explainable AI techniques which may require additional processing time or resources and so may take more time to complete. Explainable AI is a technique for extracting and/or visualizing the correlation or importance of input variables to the system outputs. Additionally or alternatively, the slow processing may be used to train or update a model which may be subsequently applied to buffered data. Further, the relatively slow processing may be used to adjust or change a processing that may be monitored in real time. For example, real-time imaging may be used while performing a treatment such as laser treatment. The relatively slow processing may be used to evaluate how the treatment is progressing and possibly adjust further treatment based on the results. The adjustment may include varying treatment parameters such as laser power, pulse durations, etc. and/or may include varying treatment locations or possibly suggesting further treatments that may be beneficial to perform during the same treatment process.

The above has described processing images of a patient's eye, however the hybrid processing could be applied to processing images of other parts of a patient. Further, the above has described a treatment process using lasers however other treatment options are possible using for example robotic surgery techniques.

The above has described the processing being applied to images, however it may be applied to different types of data, such as sensor data, audio data etc. As an example, a real-time audio processing technique may separate and perform speech to text on audio from different speakers. An audio processing model may be used to identify different speakers present in the audio being captured; however, the model may need to first be trained in order to identify the different speakers. The model may be trained while audio data is captured and stored in the buffer. Once the audio model is trained it can be applied to the buffered audio data.

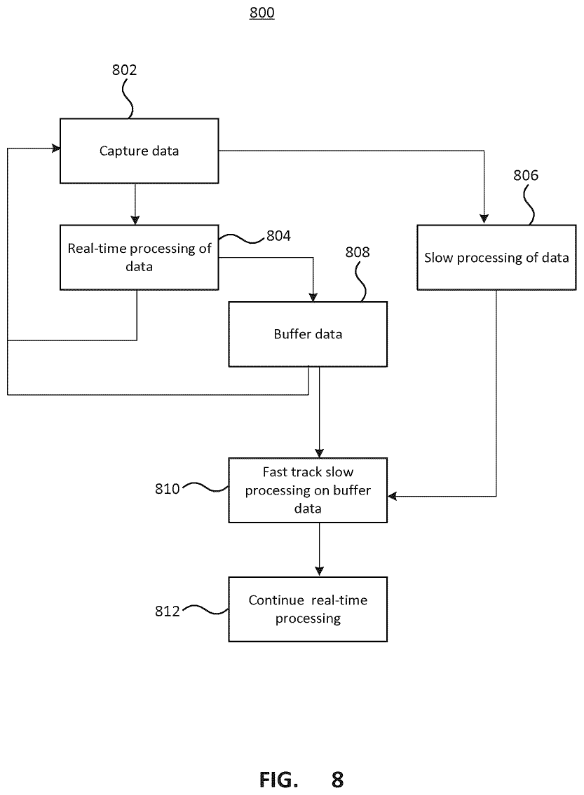

FIG. 8 depicts a method of hybrid data processing. The method 800 is similar to the methods described above, but may be applied to areas other than image processing. The method 800 captures data (802). The data being is captured at a capture rate. The data may be image data as described above, or may be other types of data such as audio data, sensor data, etc. A real-time processing techniques is applied to the captured data (804). The real-time processing of the data may be a process that is applied to frame or portion of the data in a length of time that is equal to or shorter than the length of time of the frame or portion of data. As the data is being processed in real-time, a relatively slow, or non-real-time process may be applied to the data (806). The non-real-time processing of the data may include identifying features within the data, fusing the data with other data sources, applying machine learning models to the data, and/or training models to be applied to the data. While the non-real-time processing occurs, the data being captured and processed in real-time is also buffered (808). It is possible that the real-time processing may only occur after the non-real-time processing has occurred. For example, if the non-real-time processing identifies some features within the captured data, and the real-time processing tracks the identified features, it may not be possible to track the features in real-time until the features have been identified using the non-real-time processing.

Once the non-real-time processing of the captured data is completed, the processing results may be applied to the buffered data, which may be considered as fast-tracking the non-real-time processing results across the buffered data (810). It will be appreciated that different data types and different real-time and non-real-time processing may apply the slow processing results to the buffered data in various ways, such as possibly tracking an identified feature across the buffered data, applying a model to the buffered data, modify the buffered data according to the slow processing results, etc.

Once the non-real-time processing results have been fast-tracked across the buffered data, the real-time processing may continue (812). The data may continue to be buffered so that if subsequent slow processing is necessary, or desired, it may be performed and then again applied to the buffered data.

As an example of possible hybrid processing provided using both real-time and non-real-time processing, it may be possible to perform laser treatment for glaucoma on a target using an imaging and laser system with real-time tracking and ML processing. Additional sensors, such as an IOP sensor, may read the internal eye pressure and an ML algorithm may be running monitor the pressure and determine treatment options, such as continuing treatment, providing a further treatment target, or stopping treatment.

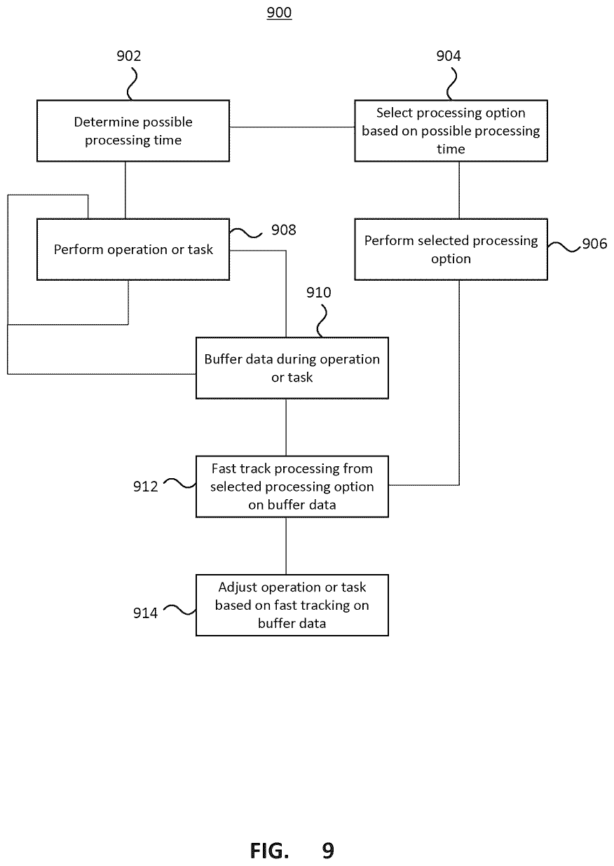

FIG. 9 depicts a further method of hybrid data processing. The method 900 is similar to the methods described above, however may change the non-real-time processing technique applied to the data. The method 900 determines a possible processing time (902), which may then be used to select a processing option based on the possible processing time (904). The selected processing option is performed (906), which is a non-real-time process. The possible processing time may be an amount of time that the non-real-time processing technique may use to complete the processing. For example, an operation or task may be performed (908) that may take a certain length of time. If the operation or task is a treatment of a condition, the possible time may be an estimate of how long the procedure will take. The time estimate may be determined using previous data, or possibly estimated using the patient's data. During the operation or task the data is buffered (910). Once the non-real-time processing option is completed, the processing result from the selected non-real-time processing option may be applied to the buffered data (912). The operation or task may then be adjusted based on the fast tracking of the non-real-time processing across the buffered data.

The method 900 may be used to possibly apply more complex data processing to the data when the time is available. The more complex data processing may provide additional results, or possibly improved results. As an example, if the operation or task is a treatment process that takes 5 seconds to complete the possible processing that may be applied to the captured data may be less than the processing that may be applied to the data if the treatment process is expected 1 minute to complete. Given the longer processing time, additional processing may be provided such as evaluating a treatment result, such as whether or not an initial portion of the treatment may be considered successful or possibly unsuccessful and so require additional treatment. The results, of the non-real-time processing may then be applied across the buffered data, for example by tracking a location of an area of successful treatment or area requiring further treatment. Although not depicted in FIG. 9, it is possible for the processing results to be presented to a user or operator, for example by presenting them with options for further processing based on the non-real-time results.

It will be appreciated by one of ordinary skill in the art that the system and components shown in FIGS. 1-9 may include components not shown in the drawings. For simplicity and clarity of the illustration, elements in the figures are not necessarily to scale, are only schematic and are non-limiting of the elements structures. It will be apparent to persons skilled in the art that a number of variations and modifications can be made without departing from the scope of the invention as defined in the claims.

Although certain components and steps have been described, it is contemplated that individually described components, as well as steps, may be combined together into fewer components or steps or the steps may be performed sequentially, non-sequentially or concurrently. Further, although described above as occurring in a particular order, one of ordinary skill in the art having regard to the current teachings will appreciate that the particular order of certain steps relative to other steps may be changed. Similarly, individual components or steps may be provided by a plurality of components or steps. One of ordinary skill in the art having regard to the current teachings will appreciate that the components and processes described herein may be provided by various combinations of software, firmware and/or hardware, other than the specific implementations described herein as illustrative examples.

The techniques of various embodiments may be implemented using software, hardware and/or a combination of software and hardware. Various embodiments are directed to apparatus, e.g. a node which may be used in a communications system or data storage system. Various embodiments are also directed to non-transitory machine, e.g., computer, readable medium, e.g., ROM, RAM, CDs, hard discs, etc., which include machine readable instructions for controlling a machine, e.g., processor to implement one, more or all of the steps of the described method or methods.

Some embodiments are directed to a computer program product comprising a computer-readable medium comprising code for causing a computer, or multiple computers, to implement various functions, steps, acts and/or operations, e.g. one or more or all of the steps described above. Depending on the embodiment, the computer program product can, and sometimes does, include different code for each step to be performed. Thus, the computer program product may, and sometimes does, include code for each individual step of a method, e.g., a method of operating a communications device, e.g., a wireless terminal or node. The code may be in the form of machine, e.g., computer, executable instructions stored on a computer-readable medium such as a RAM (Random Access Memory), ROM (Read Only Memory) or other type of storage device. In addition to being directed to a computer program product, some embodiments are directed to a processor configured to implement one or more of the various functions, steps, acts and/or operations of one or more methods described above. Accordingly, some embodiments are directed to a processor, e.g., CPU, configured to implement some or all of the steps of the method(s) described herein. The processor may be for use in, e.g., a communications device or other device described in the present application.

Numerous additional variations on the methods and apparatus of the various embodiments described above will be apparent to those skilled in the art in view of the above description. Such variations are to be considered within the scope.

What is claimed is:

1. An image processing method comprising:
receiving a first image of an image stream having a stream frame rate;
passing the received first image to image processing functionality;
receiving a plurality of subsequent images of the image stream;
storing the received plurality of subsequent images in a fast-track image buffer;
subsequent to receiving at least one subsequent image, receiving from the image processing functionality an indication of one or more features within the first image; and
tracking the one or more features across the plurality of subsequent images stored within the fast-tracking image buffer using a tracking process configured to process each of the subsequent images at a processing frame rate higher than the stream frame rate.

2. The method of claim 1, wherein the image processing functionality identifies the one or more features in the received first image.

3. The method of claim 1, wherein the image processing functionality identifies the one or more features in the received first image using a machine learning process.

4. The method of claim 1, wherein passing the received first image to the image processing functionality comprises:
passing the first image to the processing functionality implemented at a remote computing device over a communication interface.

5. The method of claim 1, further comprising:
after tracking the one or more features across all of the subsequent images stored in the fast-tracking buffer, tracking the one or more features across newly received images of the image stream.

6. The method of claim 5, wherein tracking the one or more features across the newly received images of the image stream uses the tracking process used for tracking the one or more features across the subsequent images stored within the fast-tracking image buffer.

7. The method of claim 5, wherein tracking the one or more features across the newly received images of the image stream uses a different tracking process than used for tracking the one or more features across the subsequent images stored within the fast-tracking image buffer, wherein the tracking process for tracking the one or more features across the newly received images of the image stream has a slower processing frame rate than the processing frame rate of the tracking process used for tracking the one or more features across the subsequent images stored within the fast-tracking image buffer.

8. The method of claim 1, further comprising:
receiving new images of the image stream while tracking the one or more features across the subsequent images stored within the fast-tracking image buffer; and
storing the new images in the fast-tracking image buffer.

9. The method of claim 1, further comprising one or more of:
removing subsequent images stored in the fast-tracking image buffer once processed by the tracking process; and
marking subsequent images stored in the fast-tracking image buffer as safe for removal once processed by the tracking process.

10. The method of claim 1, wherein image stream is of a patient's eye, the method further comprising:
using the one or more identified features tracked across images of the image stream in treatment of an eye condition.

11. An image processing device comprising:
a processor capable of executing instructions; and
a memory storing instructions which when executed by the processor configure the image processing device to provide a method comprising:
receiving a first image of an image stream having a stream frame rate;
passing the received first image to image processing functionality;
receiving at least one subsequent image of the image stream;
storing the received at least one subsequent images in a fast-tracking image buffer;
subsequent to receiving at least one subsequent image, receiving from the image processing functionality an indication of one or more features within the first image; and
tracking the one or more features across the subsequent images stored within the fast-tracking image buffer using a tracking process capable of processing the subsequent images at a processing frame rate higher than the stream frame rate.

12. The image processing device of claim 11, wherein the image processing functionality identifies the one or more features in the received first image.

13. The image processing of claim 11, wherein the image processing functionality identifies the one or more features in the received first image using a machine learning process.

14. The image processing device of claim 11, further comprising a communication interface for communicating with a remote computing device, the method provided by execution of the instructions comprises:
passing the first image to the processing functionality implemented by the remote computing device over the communication interface.

15. The image processing device of claim 14, wherein the communication interface is at least one of:
a PCIe communication interface;
a USB interface;
a Bluetooth interface;
a wired network interface; and
a wireless network interface.

16. The image processing device of claim 11, wherein the method provided by executing the instructions further comprises:
after tracking the one or more features across all of the subsequent images stored in the fast-tracking buffer, tracking the one or more features across newly received images of the image stream.

17. The image processing device of claim 16, wherein tracking the one or more features across the newly received images of the image stream uses a same tracking process as used for tracking the one or more features across the subsequent images stored within the fast-tracking image buffer.

18. The image processing device of claim 16, wherein tracking the one or more features across the newly received images of the image stream uses a different tracking process than used for tracking the one or more features across the subsequent images stored within the fast-tracking image buff, wherein the tracking process for tracking the one or more features across the newly received images of the image stream has a slower processing frame rate than the processing frame rate of the tracking process used for tracking the one or more features across the subsequent images stored within the fast-tracking image buffer.

19. The image processing device of claim 11, wherein the method provided by executing the instructions further comprises:
receiving new images of the image stream while tracking the one or more features across the subsequent images stored within the fast-tracking image buffer; and
storing the new images in the fast-tracking image buffer.

20. The image processing device of claim 11, wherein the method provided by executing the instructions further comprises one or more of:
removing subsequent images stored in the fast-tracking image buffer once processed by the tracking process; and
marking subsequent images stored in the fast-tracking image buffer as safe for removal once processed by the tracking process.

21. The method of claim 11, wherein image stream is of a patient's eye, the method further comprising:
using the one or more identified features tracked across images of the image stream in treatment of an eye condition.
